SEQUENCE LISTING (2 sequences)

SEQ ID NO: 1 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - protein, 192 aa, Homo sapiens
MRGSHHHHHHGSMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

(19) United States
(12) Patent Application Publication
(10) Pub. No.: US 2014/0171626 A1
(43) Pub. Date: Jun. 19, 2014

(54) PROTEIN PURIFICATION

(71) Applicants: Louis Patrick GAGNON, Rixensart (CA); John Williams, Rixensart (CA)

(72) Inventors: Louis Patrick GAGNON, Rixensart (CA); John Williams, Rixensart (CA)

(73) Assignee: GlaxoSmithKline LLC, Philadelphia, PA (US)

(21) Appl. No.: 13/791,021

(22) Filed: Mar. 8, 2013

Related U.S. Application Data
(60) Provisional application No. 61/739,149, filed on Dec. 19, 2012.

(51) Int. Cl. C07K 14/47

(52) U.S. Cl. CPC C07K 14/4748
USPC 530/413; 530/350

(57) ABSTRACT

Processes for the purification of NY-ESO-1 related polypeptides, as well as polypeptides produced by these processes are provided herein; a composition comprising pure and stable NY-ESO-1 is also provided.

### CROSS REFERENCE TO RELATED APPLICATIONS

This application under 35 USC Section 111(a) claims the benefit of U.S. provisional application Ser. No. 61/739,149 filed Dec. 19, 2012.

### FIELD OF THE INVENTION

The present invention relates generally to a pure and stable NY-ESO-1 related polypeptide and processes for producing the same.

### BACKGROUND TO THE INVENTION

The gene regulation of a class of proteins generally expressed only in tumour cells and immune privileged tissues, termed cancer-testis antigens (CT antigens), is disrupted in cancer patients. This leads to the aberrant expression of these antigens in a wide variety of tumours. The immunogenicity of some CT antigens in cancer patients makes them an ideal target for the development of tumour vaccines. A cancer testis antigen currently of interest for use in cancer immunotherapy is NY-ESO-1. A further cancer testis antigen, LAGE-1, has also been identified. Two LAGE-1 transcripts have been described, LAGE-1a and LAGE1b. LAGE-1b is incompletely spliced and codes for a putative protein of approximately 210 amino acids residues, while the LAGE-1a gene product contains 180 amino acid residues (Sun et al. (2006) Cancer Immunol Immunother, 6:55:644-652).

The N-terminal regions of the LAGE-1 and NY-ESO-1 proteins are highly conserved and are thought to have more than 97% identity. However, LAGE-1 differs from NY-ESO-1 in the central regions which are only 62% identical. The C-terminals of NY-ESO-1 and LAGE-1a are highly conserved (more than 97% identity). However, the C-terminal of LAGE-1b is longer and not conserved and is thought to have less than 50% identity with the same region in LAGE-1a/NY-ESO-1. General information relating to these proteins is available from the LICR web site (see www.cancerimmunity.org/CTdatabase).

The purification of recombinant CT antigens is one step involved in the manufacture of cancer immunotherapeutics based on CT antigens. Various approaches have been investigated to remove contaminants such as host cell proteins from recombinantly produced proteins and kits are commercially available to aid the development of processes for producing soluble and active recombinant proteins from host cells such as E. coli expression systems. See, e.g., “Tools for Enhancing Solubility of Proteins Expressed in E. coli and refolding screens for Difficult Targets, copyright 2008 EMD Chemicals Inc., an affiliate of Merck KGaA, Damstadt, Germany. Nonetheless, in some cases difficulties have been encountered in achieving sufficient purity levels for CT antigens.

For instance, the production of recombinant NY-ESO-1 has proven difficult to produce using standard E. coli-based recombinant expression systems. See Murphy (2005) Prep. Biochem. Biotechnol. 35:119-134. Such purification schemes involve multiple chromatography steps with a concomitantly high loss of the NY-ESO-1 protein. In addition, the low levels of purity obtained have given rise to concerns over the immunotherapeutic use of recombinant NY-ESO-1 purified according to the published method due to possible delayed-type hypersensitivity reactions caused by contaminating E. coli proteins. Efforts to resolve these issues have led to the use of recombinant NY-ESO-1 variants mutated for compatibility with alternate expression systems such as yeast. See Piatesi (2006) Protein Expression and Purification 48:232-242.

### SUMMARY OF THE INVENTION

Processes for the purification of NY-ESO-1 related polypeptides, as well as polypeptides produced by these processes are provided herein. A composition comprising pure and stable NY-ESO-1 is also provided.

In some embodiments are provided a process for purifying a NY-ESO-1 related polypeptide having an affinity tag comprising the steps of (a) solubilizing a composition comprising the polypeptide having an affinity tag in a buffer comprising N-Lauroylsarcosine and TCEP to obtain a mixture; and (b) purifying the mixture by immobilized metal affinity chromatography (IMAC) to obtain pure polypeptide having an affinity tag.

In some embodiments, a suitable buffer comprises 20 mM Tris pH 8.0, 5% N-Lauroylsarcosine, 20 mM Imidazole, and 5 mM TCEP. In some embodiments, the composition is a cell pellet. In some embodiments, the affinity tag is a metal ion binding affinity tag. In some embodiments, the affinity tag is a his tag.

In some embodiments are provided a process for purifying a NY-ESO-1 related polypeptide having an affinity tag comprising the steps of (a) solubilizing a composition comprising the polypeptide having an affinity tag in a buffer comprising N-lauroylsarcosine and TCEP to obtain a mixture; (b) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP to obtain pure polypeptide having an affinity tag.

In some embodiments, the IMAC substrate is Ni²⁺. In some embodiments, the IMAC substrate is contained in a column. In some embodiments, the first buffer comprises 20 mM Tris pH 8, 1% N-Lauroylsarcosine, and 1 mM TCEP. In some embodiments, the second buffer comprises 20 mM Tris pH 7, 8 M Urea, 0.5 M NaCl, 1 mM TCEP, and 20 mM Imidazole. In some embodiments, the elution buffer comprises 20 mM Tris pH 7, 8 M Urea, 0.5 M NaCl, 1 mM TCEP, and 500 mM imidazole.

In some embodiments are provided a process for purifying a NY-ESO-1 related polypeptide having an affinity tag comprising the steps of (a) solubilizing a composition comprising the polypeptide having an affinity tag in a buffer comprising N-Lauroyl sarcosine and TCEP to obtain a mixture; (b) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP; (e) subjecting the polypeptide having an affinity tag to buffer exchange to obtain pure polypeptide having an affinity tag.

In some embodiments, the buffer exchange step is carried out using chromatography on a sephadex G-25 column. In some embodiments, the buffer exchange is carried out using a buffer comprising 10 mM sodium acetate pH 5.

In some embodiments are provided a process for purifying a NY-ESO-1 related polypeptide having an affinity tag comprising the steps of (a) solubilizing a composition comprising the polypeptide having an affinity tag in a buffer comprising N-Lauroyl sarcosine and TCEP to obtain a mixture; (b) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP; (c) subjecting the polypeptide having an affinity tag to buffer exchange to obtain an eluate comprising the polypeptide having an affinity tag; and (d) concentrating and diafiltrating the polypeptide having an affinity tag to obtain pure polypeptide having an affinity tag.

In some embodiments, the concentrating and diafiltrating step is done by tangential flow filtration (TFF). In some embodiments, the concentration and diafiltration is done on a 10 kDa regenerated cellulose C-screen membrane in a buffer. In some embodiments, the concentration and diafiltration is done in a buffer comprising 10 mM sodium acetate pH 5.

In some embodiments are provided a process for purifying a NY-ESO-1 related polypeptide having an affinity tag comprising the steps of (a) solubilizing a composition comprising the polypeptide having an affinity tag in a buffer comprising N-Lauroyl sarcosine and TCEP to obtain a mixture; (b) clarifying the mixture; (c) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP; (d) subjecting the polypeptide having an affinity tag to buffer exchange to obtain an eluate comprising the polypeptide having an affinity tag; and (e) concentrating and diafiltrating the polypeptide having an affinity tag to obtain pure polypeptide having an affinity tag.

In some embodiments, the clarification step further comprises centrifuging the mixture. In some embodiments, the clarification step further comprises filtration of the mixture. In some embodiments, the clarification step further comprises both centrifuging the mixture and filtration of the mixture. In some embodiments, the centrifuging step occurs at 15,900×g. In some embodiments, the filtration step occurs using a 0.45/0.22 um PES filter.

In some embodiments are provided a process for purifying a NY-ESO-1 related polypeptide having an affinity tag comprising the steps of (a) disrupting cells comprising the polypeptide having an affinity tag to obtain a composition; (b) solubilizing the composition in a buffer comprising N-Lauroyl sarcosine and TCEP to obtain a mixture; (c) clarifying the mixture; (d) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP; (e) subjecting the polypeptide having an affinity tag to buffer exchange to obtain an eluate comprising the polypeptide having an affinity tag; and (f) concentrating and diafiltrating the polypeptide having an affinity tag to obtain pure polypeptide having an affinity tag.

In some embodiments are provided a process for purifying a NY-ESO-1 related polypeptide having an affinity tag comprising the steps of (a) disrupting cells comprising the polypeptide having an affinity tag to obtain a composition; (b) solubilizing the composition in a buffer comprising N-Lauroyl sarcosine and TCEP to obtain a mixture; (c) clarifying the mixture; (d) purifying the mixture by IMAC comprising the substeps of (i) loading the mixture on an IMAC substrate; (ii) washing the substrate with a first buffer comprising N-Lauroylsarcosine and TCEP; (iii) washing the substrate with a second buffer comprising Urea, NaCl, and TCEP; (iv) eluting the polypeptide having an affinity tag with an elution buffer comprising Urea, NaCl, and TCEP; (e) subjecting the polypeptide having an affinity tag to buffer exchange to obtain an eluate comprising the polypeptide having an affinity tag; (f) concentrating and diafiltrating the polypeptide having an affinity tag; and (g) subjecting the polypeptide having an affinity tag to filtration to obtain pure polypeptide having an affinity tag.

In some embodiments, the filtration is sterile filtration. In some embodiments, the filtration is done on a 0.45/0.2 Sartobran™ 300 cellulose acetate capsule.

In some embodiments are provided a process for purifying a NY-ESO-1 related polypeptide having a his tag comprising the steps of (a) disrupting cells comprising the polypeptide having a his tag by the substeps comprising (i) suspending the cells in a buffer comprising 50 mM Tris pH 8, 150 mM NaCl, and 10 mM EDTA; (ii) applying a cell disruption technique, for example, high pressure homogenizer, French press, or osmotic shock; (iii) centrifuging the cells at 15,900×g to obtain a pellet; (iv) washing the pellet in a buffer comprising 50 mM Tris pH 8 and 150 mM NaCl; (b) solubilizing the pellet in a buffer comprising 20 mM Tris pH 8.0, 5% N-Lauroylsarcosine, 20 mM Imidazole, and 5 mM TCEP to obtain a mixture; (c) clarifying the mixture by the substeps of (i) centrifuging the mixture at 15,900×g to obtain a supernatant; (ii) filtration of the supernatant through a 0.45/0.22 um PES filter; (d) purifying the mixture by IMAC comprising the substeps of (i) loading the mixture on a Ni²⁺-IMAC sepharose column; (ii) washing the column with a buffer comprising 20 mM Tris pH 8, 1% N-Lauroylsarcosine, and 1 mM TCEP; (iii) washing the column with a buffer comprising 20 mM Tris pH 7, 8 M Urea, 0.5 M NaCl, 1 mM TCEP, and 20 mM Imidazole; (iv) eluting the polypeptide having a his tag with a buffer comprising 20 mM Tris pH 7, 8 M Urea, 0.5 M NaCl, 1 mM TCEP and 500 mM imidazole to obtain an eluate comprising the polypeptide having a his tag; (e) subjecting the polypeptide having a his tag to buffer exchange chromatography on a sephadex G-25 column using a buffer comprising 10 mM sodium acetate pH 5 to obtain an eluate comprising the polypeptide having a his tag; (f) concentrating and diafiltrating the polypeptide having a his tag by TFF on a 10 kDa regenerated cellulose C-screen membrane in a buffer comprising 10 mM sodium acetate pH 5; and (g) subjecting the polypeptide having a his tag to sterile filtration on a 0.45/0.2 Sartobran™ 300 cellulose acetate capsule.

In some embodiments are provided a process for purifying a NY-ESO-1 related polypeptide having a his tag comprising the steps of (a) disrupting cells comprising the polypeptide having a his tag by the substeps comprising (i) suspending the cells in a buffer comprising 50 mM Tris pH 8, 150 mM NaCl, 10 mM EDTA; (ii) applying a cell disruption technique, for example, high pressure homogenizer, French press, or osmotic shock; (iii) centrifuging the cells at 15,900×g to obtain a pellet; (iv) washing the pellet in a buffer comprising 50 mM Tris pH 8 and 150 mM NaCl; (v) recovering the insoluble fraction containing the inclusion bodies by centrifugation and washing the pellet one time with 20 mM Tris, 5% Triton X-100, pH 8.0; (vi) recovering the insoluble fraction containing the inclusion bodies by centrifugation and washing the pellet twice with 20 mM Tris, 150 mM NaCl, pH8.0; (b) solubilizing the pellet in a buffer comprising 20 mM Tris pH 8.0, 5% N-Lauroylsarcosine, 20 mM Imidazole, and 5 mM TCEP to obtain a mixture; (c) clarifying the mixture by the substeps of (i) centrifuging the mixture at 15,900×g to obtain a supernatant; (ii) filtration of the supernatant through a 0.45/0.22 um PES filter; (d) purifying the mixture by IMAC comprising the substeps of (i) loading the mixture on a Ni²⁺-IMAC sepharose column; (ii) washing the column with a buffer comprising 20 mM Tris pH 8, 1% N-Lauroylsarcosine, and 1 mM TCEP; (iii) washing the column with a buffer comprising 20 mM Tris pH 7, 8 M Urea, 0.5 M NaCl, 1 mM TCEP, 20 mM Imidazole; (iv) eluting the polypeptide having a his tag with a buffer comprising 20 mM Tris pH 7, 8 M Urea, 0.5 M NaCl, 1 mM TCEP, and 500 mM imidazole to obtain an eluate comprising the polypeptide having a his tag; (e) subjecting the polypeptide having a his tag to buffer exchange chromatography on a sephadex G-25 column using a buffer comprising 10 mM sodium acetate pH 5 to obtain an eluate comprising the polypeptide having a his tag; (f) concentrating and diafiltrating the polypeptide having a his tag by TFF on a 10 kDa regenerated cellulose C-screen membrane in a buffer comprising 10 mM sodium acetate pH 5; and (g) subjecting the polypeptide having a his tag to sterile filtration on a 0.45/0.2 Sartobran™ 300 cellulose acetate capsule.

In some embodiments are provided a composition comprising polypeptides, wherein said polypeptides comprise by w/w at least 95% monomer+dimer+trimer species of a polypeptide comprising the amino acid sequence of SEQ ID NO:1. In some embodiments are provided a composition comprising a polypeptide comprising the amino acid sequence of SEQ ID NO:1, wherein the composition further comprises no more than 5% host cell protein content. In some embodiments are provided a composition comprising a polypeptide comprising the amino acid sequence of SEQ ID NO:1, wherein the composition further comprises a low complexity solution.

In some embodiments, the polypeptide comprises one or more of the following characteristics selected from the group consisting of: (a) soluble polypeptide aggregates with hydrodynamic size of about 22 nm; (b) concentration of at least about 2 mg/mL; (c) weight averaged molecular weight of about 640 kDa; (d) positive Zeta-potential of about 23 mV; (e) 100% free cystein as determined by MS; and (f) low complexity solution comprising 10 mM Na-Ac pH 5.0. In some embodiments, the NY-ESO-1 polypeptide (SEQ ID NO:1) forms about 77% of monomer; 14% dimer; and 2.5% trimer as measured by SDS-PAGE.

In some embodiments are provided a composition comprising N-Lauroyl sarcosine; TCEP; and one or more cancer testes antigens comprising full-length NY-ESO-1 protein. In some embodiments the full-length NY-ESO-1 protein comprises an amino acid sequence selected from the group comprising SEQ ID NOS:1-2. In some embodiments, the full-length NY-ESO-1 protein further comprises a fusion partner or a carrier protein.

### BRIEF DESCRIPTION OF THE FIGURES

FIG. 1/4. A flow chart describing the fermentation process for the NY-ESO-1 construct is shown.

FIG. 2/4. A flow chart for the His/NY-ESO-1 purification is shown.

FIG. 3/4. Coomasie blue stained SDS-PAGE comparing the polypeptide produced by three separate runs using the disclosed process. Legend (by lane, left to right): 1. Molecular weight marker; 2. P-036 DNYEAHA005 (8 μg); 3. P-037 DNYEAHA006 (8 μg); and 4. P-038 DNYEAHA007 (8 μg).

FIG. 4/4. NY-ESO-1 polypeptide produced in three separate runs was analyzed by Western blot analysis using polyclonal antibody to α-E. coli BLR DE3 proteins. Legend (by lane, left to right): (1) E. coli BL21 DE3 extract, 20 μg; (2) E. coli BL21 DE3 extract, 5 μg; (3) E. coli BL21 DE3 extract, 2 μg; (4) E. coli BL21 DE3 extract; 1 μg; (5) P-036-DNYEAHA005, 20 μg; (6) P-036-DNYEAHA005, 20 μg+1 μg HCP spike (5% contamination); (7) P-037-DNYEAHA006, 20 μg; (8) P-037-DNYEAHA006, 20 μg+1 μg HCP spike (5% contamination); (9) P-038-DNYEAHA007, 20 μg; (10) P-038-DNYEAHA007, 20 μg+1 μg HCP spike (5% contamination).

### DETAILED DESCRIPTION

### Definitions

“NY-ESO-1 related polypeptides” are defined elsewhere herein.

“Pure” or “purity level” when used in the context of NY-ESO-1 related polypeptides means an NY-ESO-1 related polypeptide composition in which at least a given percent of the total polypeptide therein is the NY-ESO-1 related polypeptide, as determined by the chosen assay method. In some embodiments, the purity level of an NY-ESO-1 related polypeptide is determined using NuPAGE 4-12% Bis-Tris gel, Coomassie Blue G-250, under reducing conditions (available from Invitrogen Corporation, 5791 Van Allen Way, PO Box 6482, Carlsbad, Calif. 92008).

Commercially available BSA is utilized to generate a standard curve and commercially available scanning densiometry, such as ImageQuant Software, is used for densiometry analysis (available from GE Healthcare). In some embodiments, an NY-ESO-1 related polypeptide composition produced according to the process disclosed herein comprises at least 95%, 96%, 97%, 98%, 99%, 99.5% NY-ESO-1 related polypeptide monomer+dimer+trimer species, w/w. In some embodiments, an NY-ESO-1 polypeptide composition comprises at least 60%, 65%, 70%, 75%, 80%, or more, monomeric NY-ESO-1 species, w/w.

“Host cell protein content” when used in the context of NY-ESO-1 related polypeptides means the percent of host cell protein detected in an NY-ESO-1 related polypeptide composition using the chosen assay. In some embodiments, host cell protein content is determined by Western blot using NuPAGE 4-12% Bis-Tris gel, under reducing conditions and using anti-E coli antibodies against specific strain of host cell. The antibodies may be monoclonal or polyclonal and may be produced using routine methods or purchased commercially. In some embodiments, an NY-ESO-1 related polypeptide composition comprises no more than 5%, 4%, 3%, 2%, 1%, 0.5%, 0.4%, 0.3%, 0.2%, 0.1% host cell protein content, as determined by the selected assay method. In some embodiments, an NY-ESO-1 related polypeptide composition produced according to the process disclosed herein comprises less than 5%, 4%, 3%, 2%, 1%, 0.5%, 0.4%, 0.3%, 0.2%, 0.1% host cell protein content.

“Low complexity solution” as used herein refers to a solution comprising one or more excipients at a low concentration. Generally, a low complexity solution will comprise no more than 100 mM, 90 mM, 80 mM, 70 mM, 60 mM, 50 mM, 40 mM, 30 mM, 20 mM, 10 mM, 5 mM, 4 mM, 3 mM, 2 mM, or 1 mM of any given excipient, although a low complexity solution may comprise more than one excipient.

### Manufacturing

The general approach for producing NY-ESO-1 related polypeptides involves the following processes:

1. Preparation of a recombinant host cell strain expressing the affinity tagged NY-ESO-1 polypeptide;

2. Preparation of a seed system (Parent, Master and Working Seed Banks) derived from this strain;

3. Fermentation of the recombinant host cells in a 20-L-fermentor;

4. Disruption of host cells and extraction of a crude composition containing the NY-ESO-1 related polypeptide;

5. Extraction and purification of NY-ESO-1 related polypeptide from the crude composition.

### Disruption, Extraction, and Purification

Cell Disruption

Post-induction, host cells containing the NY-ESO-1 related polypeptide may be isolated and disrupted by routine methods, such as high pressure homogenization, French press, or osmotic shock, etc. The disrupted cellular debris, containing the NY-ESO-1 related polypeptide, can be isolated by various means, including centrifugation, TFF, vibrating membrane (as disclosed in U.S. Pat. No. 6,939,697 to Permanne et al).

In some embodiments, host cells are disrupted by the steps comprising:

1. Suspending the host cells in a buffer comprising 50 mM Tris pH 8, 150 mM NaCl, 10 mM EDTA;

2. Host cell disruption by, for instance, high pressure homogenizer;

3. Centrifuging the host cells at 15,900×g to obtain a pellet;

4. Washing the pellet in a buffer comprising 50 mM Tris pH 8 and 150 mM NaCl.

Wash.

In some embodiments, the insoluble fraction containing the inclusion bodies is recovered by centrifugation and the pellet washed one time with 20 mM Tris, 5% Triton X-100, pH 8.0. The insoluble fraction containing the inclusion bodies is recovered by centrifugation and the pellet washed twice times with 20 mM Tris, 150 mM NaCl, pH8.0.

Solubilization.

After disruption (and optionally the wash step), the isolated cellular debris containing the NY-ESO-1 related polypeptide is solubilized. In some embodiments, the isolated cellular debris is solubilized in a buffer comprising N-lauroylsarcosine and Tris (2-carboxyethyl) phosphine hydrochloride (TCEP). In some embodiments, the isolated cellular debris is solubilized in a buffer comprising 5% N-lauroylsarcosine and 5 mM TCEP. In some embodiments, the isolated cellular debris is solubilized in a buffer comprising 20 mM Tris pH 8.0, 5% N-lauroylsarcosine, 20 mM Imidazole, and 5 mM TCEP.

Clarification.

After the cellular debris containing the NY-ESO-1 related polypeptide is solubilized, a clarification step may be carried out by a variety of techniques including centrifugation, filtration, etc. In some embodiments, clarification is carried out using a commercially available 0.45/0.22 μm poyethersulfone (PES) dual cut-off filter. In some embodiments, clarification is carried out by centrifugation. In some embodiments, clarification by centrifugation is carried out at 15,900×g.

Immobilized Metal Ion Affinity Chromatography (IMAC).

The purification of recombinant proteins has been simplified by affinity chromatographic strategies in which a peptide or protein-affinity tag, cloned in-frame with the target construct, selectively interacts with a ligand that has been immobilized on a solid support. Immobilized metal ion affinity chromatography (IMAC), one embodiment of this methodology, relies on divalent transition metal ions (such as Ni²⁺, Zn²⁺, Co²⁺, Cu²⁺, Mn²⁺, or Mg²⁺) which are immobilized by surface-bound chelators such as iminodiacetic acid (IDA) or nitrilotriacetic acid (NTA). The protein to be purified interacts with the free coordination sites of the immobilized transition metal ions. See, e.g., Lichty et al. (2005) Protein Expression and Purification 41:98-105; Knecht (2008) J. Mol. Recognit. 22:270-279. IMAC media comprising Ni²⁺ immobilized by chelation with nitrilotriacetic acid (NTA) bound to a solid support has become a common method for the purification of proteins carrying either a C- or N-terminal metal ion chelation affinity tag. In some embodiments, the media is a Ni²⁺ sepharose media. Such media is commercially available from GE Healthcare, a division of General Electric Company.

Common His-tag technology typically comprising five to six consecutive His residues was used. Since it is rather rare that such oligohistidine segments are expressed in naturally occurring proteins, 5His- or 6His-tags usually result in high selectivity. Knecht (2008) J. Mol. Recognit. 22:270-279. Nonetheless, other length His-tags may be used, comprising 1, 2, 3, 4, 5, 6, 7, 8, 9, 10 or more consecutive His residues. In some embodiments, residues other than His are included in the tag. See, e.g., Knecht (2008) J. Mol. Recognit. 22:270-279; Charlton (2008) Methods in Molecular Biology 421:25-36. In some embodiments, the His-tag may comprise (Hisₓ-R_{y})_{z}, where R is a non-His residue and x, y, and z are positive integers. In some embodiments, a HAT-tag may be used. See Terpe (2003) Appl. Microbiol. Biotechnol. 60:523-533. In some embodiments, a His-tag may be used in conjunction with one or more different affinity tags. In some embodiments, a His-tag may be located at or near the N-terminus of the protein. In other embodiments, as His-tag may be located at or near the C-terminus of the protein.

Chromatographic Setup

The protein to be purified may be introduced onto the solid support according to different approaches, such as batch or column chromatography. Generally, a batch chromatography approach involves the addition of an initial protein-containing composition to the solid support in a vessel using a loading buffer; this may be mixed. The solid support is then separated from the remainder and one or more wash steps can be undertaken in which a buffer is added to the solid support, then the solid support is separated from the wash buffer. Separate wash buffers may be used for the separate wash step(s). After the wash step(s), if any, an elution buffer is added to the solid support and the eluate removed from the solid support.

In a column approach, the solid support is packed into a column. The protein containing composition is then introduced onto the column in loading buffer and run through the column to allow the protein to bind to the solid support. One or more wash steps can be included in which a wash buffer or wash buffers is run through the column. An elution buffer is then run through the column and the eluate collected.

Buffers.

The traditional use of IMAC has involved the inclusion of 0.5-1 M NaCl in the first buffer to prevent the protein from interacting with the solid support on the basis of non-specific electrostatic interactions. In some embodiments, the loading buffer comprises 5% N-Lauroylsarcosine. In some embodiments, the loading buffer comprises 20 mM Tris pH 8.0, 5% N-Lauroylsarcosine, 20 mM Imidazole, and 5 mM TCEP.

Wash buffers may comprise components such as mild eluents that compete for the metal ion with histidine; non-amine salts, imidazole, non-ionic detergents, or chaotropic agents to disrupt non-specific electrostatic or hydrophobic interactions; and the pH may vary depending on the desired effect. See, e.g., Methods in Molecular Biology, vol. 421: Affinity Chromatography: Methods and Protocols, Second edition; Zachariou ed. (2007) Humana Press, Totowa, N.J.

In some embodiments, the first wash buffer comprises N-Lauroylsarcosine and TCEP. In some embodiments, the first wash buffer comprises 1% N-Lauroylsarcosine and 1 mM TCEP. In some embodiments, the first wash buffer comprises 20 mM Tris pH 8, 1% N-Lauroylsarcosine, and 1 mM TCEP.

In some embodiments, the second wash buffer comprises Urea, NaCl, and TCEP. In some embodiments, the second wash buffer comprises 8 M Urea, 0.5 M NaCl, and 1 mM TCEP. In some embodiments, the second wash buffer comprises 20 mM Tris pH 7, 8 M Urea, 0.5 M NaCl, 1 mM TCEP, and 20 mM Imidazole. In some embodiments the loading buffer comprises 20 mM Tris pH 8.0, 5% N-Lauroylsarcosine, 20 mM Imidazole, 5 mM TCEP and the second wash buffer comprises comprises 20 mM Tris pH 8.0, 5% N-Lauroylsarcosine, 20 mM Imidazole, 5 mM TCEP and 0.5M NaCl. In some embodiments, the second wash buffer comprises 20 mM Tris pH 7, 8 M Urea, 0.5 M NaCl, 1 mM TCEP, and 20 mM Imidazole.

In some embodiments, the elution buffer comprises urea, NaCl, and TCEP. In some embodiments, the elution buffer comprises 8 M Urea, 0.5 M NaCl, and 1 mM TCEP. In some embodiments, the elution buffer comprises 20 mM Tris pH 7, 8 M Urea, 0.5 M NaCl, 1 mM TCEP, 500 mM imidazole.

Buffer Exchange.

Buffer exchange may be accomplished by size exclusion chromatography on a porous resin (gel filtration or molecular sieve chromatography). When buffer exchange is carried out on a column, the column resin is generally pre-equilibrated with the buffer in which the sample is desired. The buffer containing the protein is passed through the column. The protein will not enter the resin pores and will quickly pass through. Buffer salts and other small molecules will enter the pores of the resin, slowing their rate of migration through the resin bed. The reduction in flow rate cases the faster macromolecules to become separated from the slower, smaller molecules. By collecting separate fractions as they emerge from the column, the macromolecule of interest can be recovered separate from the small molecules which exit the bed later. Thus, the buffer constituents carrying the sample into the column will be replaced by the solution with which the column is pre-equilibrated. Acceptable resins may comprise cellulose, polyacrylamide, dextran, etc., so long as the protein is not retained within the resin. Columns comprising appropriate resins are commercially available and include G-10, G-15, and G25 sephadex. In some embodiments, buffer exchange is carried out using G-25 sephadex resin. In some embodiments, the buffer in which the protein is collected comprises 10 mM sodium acetate pH 5. Other buffers in which NY-ESO-1 is soluble include the following:
10 mM Na-Acetate pH 5.0, 1 mM EDTA, 1 mM TCEP, 5 mM NaCl
10 mM Na-PO4 pH 6.0 or 6.5, 1 mM EDTA, 1 mM TCEP, 500 mM NaCl
10 mM Na-PO4 pH 6.5, 1 mM EDTA, 250 mM Arginine 1 mM TCEP, 250 mM NaCl
10 mM Na-PO4 pH 6.5 or 7.0, 1 mM EDTA, 300 mM Arginine, 1 mM TCEP, 5% sucrose
10 mM KPO4 pH 7.0, 350 to 500 mM Arginine
10 mM Na-PO4 pH 7.0, 1 mM EDTA, 300 mM Arginine
10 mM Na-PO4 pH 7.0, 1 mM EDTA, 300 mM Arginine, 1 mM TCEP
10 mM K-PO4 pH 7.0, 1 mM EDTA, 300 mM Arginine, 5 mM Reduced Glutathione
10 mM K-PO4 pH 7.0, 1 mM EDTA, 300 mM Arginine, 10 mM Glutamate
10 mM K-PO4 pH 7.0, 1 mM EDTA, 300 mM Arginine, 10 mM Ascorbic acid
10 mM K-PO4 pH 7.0, 1 mM EDTA, 300 mM Arginine
10 mM Tris pH 7.0, 1 mM EDTA, 1 mM TCEP, 5 mM NaCl, 250 mM Arginine, 0.15% Tween-80
   In some embodiments, other methods of buffer exchange are used, including TFF.

Concentrating; Diafiltrating.

A step of diafiltrating the protein can be included. Acceptable methods and apparati for diafiltrating include centrifugal filter devices, such as the Millipore Amicon Ultra, stirred ultrafiltration cell (available from Millipore, 290 Concord Road, Billerica, Mass. 01821) and TFF using either cassette membrane (available from Millipore) or hollow fiber type of membrane (available from 25 Harbor Park Drive, Port Washington, N.Y. 11050). At this step, the NY-ESO-1 related protein may be concentrated. TFF is suitable for this purpose.

In TFF, the fluid containing the protein of interest is pumped tangentially along the surface of a membrane. An applied pressure serves to force a portion of the fluid through the membrane to the filtrate side. Particulates and macromolecules that are too large to pass through the membrane pores are retained on the upstream side. The membrane pore size will determine which particles pass through. A variety of pore sizes may be utilized so long as the protein of interest is retained. Guidance related to TFF is available from TFF device manufacturers. See “Protein Concentration and Diafiltration by Tangential Flow Filtration,” 2003 available from Millipore Corporation, Billerica Mass., USA. Various buffers are acceptable. In some embodiments, a 10 kDa regenerated cellulose C-screen membrane is utilized to retain an NY-ESO-1 protein. In some embodiments, the diafiltration is carried out in a buffer comprising 10 mM sodium acetate pH 5. Other suitable buffers are set forth in the preceding section.

Filtration.

Following diafiltration, the solution comprising the NY-ESO-1 related polypeptide may be subjected to filtration to obtain a sterile composition. In some embodiments, a 0.45/0.2 Sartobran™ P filter may be used to filter the solution.

In some embodiments, the purified bulk protein concentration is adjusted to ˜1.7 mg/mL in 10 mM sodium acetate buffer (pH 5.0) and stored at roughly −70° C. or lower.

### NY-ESO-1 Related Polypeptides

The NY-ESO-1 related polypeptides described herein may comprise full length, partially truncated or truncated NY-ESO-1 protein or any fragment thereof that includes one or more epitopes capable of raising an immune response to NY-ESO-1.

An NY-ESO-1 protein in the context of this specification is intended to mean a protein at least 95%, 96%, 97%, 98%, 99% or 100% identical to the naturally occurring protein (SEQ ID NO:1). In some embodiments, the identity is over the entire length of the NY-ESO-1 protein sequence. Tools for determining sequence identity are widely available and include the Basic Local Alignment Search Tool (BLAST), available on the National Center for Biotechnology Information webpage at blast.ncbi.nlm.nih.gov/Blast.cgi?PROGRAM=blastp&BLAST_PROGRAMS=blastp&PAG E_TYPE=BlastSearch&SHOW_DEFAULTS=on&BLAST_SPEC=blast2seq&LINK_LOC=blasttab&LAST_PAGE=blastn&BLAST_INIT=blast2seq.

In some embodiments, NY-ESO-1 related polypeptides may differ from the naturally occurring protein by the addition, deletion, or substitution of amino acids, as described elsewhere herein. In some embodiments, the NY-ESO-1 related polypeptide includes conservative substitutions. Conservative substitutions are well known and are generally set up as the default scoring matrices in sequence alignment computer programs. These programs include PAM250 (Dayhoft M. O. et al., (1978), “A model of evolutionary changes in proteins”, “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), and “Amino acid substitution matricies from protein blocks”), Proc. Natl. Acad. Sci. USA 89 (Biochemistry): 10915-10919.

In some embodiments, an NY-ESO-1 related polypeptide is a fusion protein or chemically linked polypeptide. In some embodiments, such a fusion protein or chemically linked polypeptide comprises two or more fragments, truncated, partially truncated, or full length NY-ESO-1 proteins. In some embodiments, such a fusion protein or chemically linked polypeptide comprises heterologous polypeptide sequences. In some embodiments, the heterologous polypeptide sequence is all or a fragment of protein D from Haemophilus influenza. In some embodiments, the heterologous polypeptide sequences may be an affinity purification tag, such as a His-tag, or the equivalent, as described elsewhere herein.

Exemplary NY-ESO-1 related polypeptides include NY-ESO-1 (SEQ ID NO:1) and the NY-ESO-1 His-tag construct (SEQ ID NO:2), as described elsewhere herein. Exemplary embodiments of NY-ESO-1 related polypeptides may be found in the Sequence Listing elsewhere herein, as well as in WO2008/089074.

In some embodiments, an NY-ESO-1 related polypeptide may have a pl of roughly 8.79 without a His tag to roughly 9.10 with His tag. In some embodiments, an NY-ESO-1 related polypeptide may be present as soluble protein aggregates with hydrodynamic sizes of 20 to 25 nm. In some embodiments, an NY-ESO-1 related polypeptide may present a weight averaged molecular weight of 500-800 kDa. In some embodiments, an NY-ESO-1 related polypeptide may present a positive Zeta-potential of roughly 18 to 25 mV.

In some embodiments, a NY-ESO-1 related polypeptide presents the following characteristics:
192 amino acid recombinant protein with a pl of 9.1 (including His tag)
Theoretical MW of 19390.8 Da
5 Cys residues
   In some embodiments, a NY-ESO-1 related polypeptide solution produced by the presently disclosed process presents the following characteristics:
Soluble protein aggregates with hydrodynamic size of ≈22 nm
Concentration of ≈2 mg/mL
Weight averaged molecular weight of 640 kDa
Positive Zeta-potential of ≈23 mV
100% free cystein as determined by MS
Low complexity solution (10 mM Na-Ac pH 5.0)

Multimeric forms as determined by SDS-PAGE profile ≈77% of monomer; 14% dimer; 2.5% trimer.

In one embodiment, a NY-ESO-1 related polypeptide is termed “His/NY-ESO-1”, as described in the Examples.

### EXAMPLES

### Example 1

### Fermentation

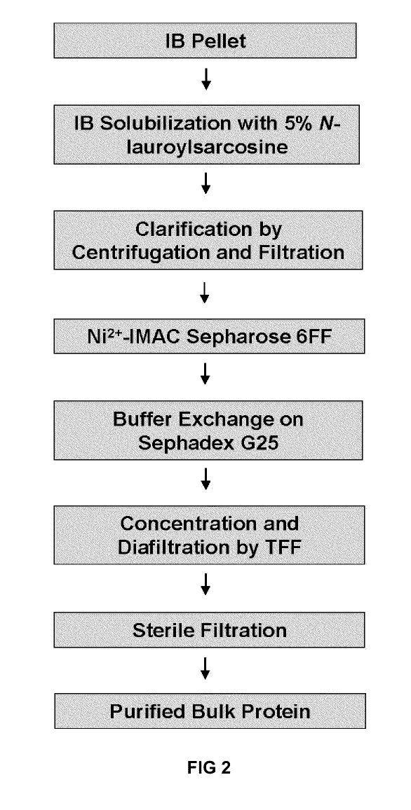

The fermentation process used to produce His/NY-ESO-1 is summarized in FIG. 2. The process is described in detail below. The compositions of the culture media are presented in m3.2.S.2.3.

m.3.2.S.2.2 Description of Manufacturing Process and Process Control

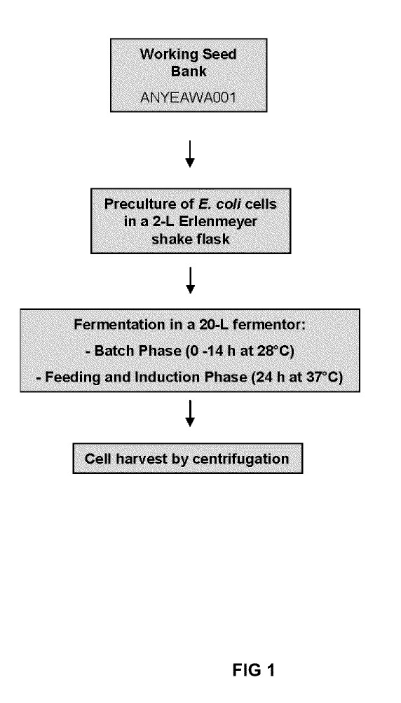

See FIG. 1/4 Summary of His/NY-ESO-1 fermentation

The current 20-L scale fermentation process is a two-step process including a liquid pre-culture stage in a shake flask followed by a fed-batch fermentation in a 20-L fermentor, including:

1 The pre-culture stage, where E. coli cells from the Manufacturer's Working Seed Bank B2270 (RIX4486; lot ANYEAWA001 from Jan. 24, 2008) harboring the recombinant plasmid pLICR100kan are grown in the presence of kanamycin and glucose at 37° C. until the value of optical density recorded at 650 nm (OD650 nm) is between 2.0 and 4.0

2 The actual fed-batch fermentation (in a 20-L fermentor) for 38±1 h, where the culture is initiated by sterile inoculation of the fermentation medium using 15 mL pre-culture (2.0<OD650 nm<4.0) from step 1; after 14 h of fermentation at 28° C., the expression of pLICR100kan-encoded product, His/NY-ESO-1, is induced with isopropyl-β-D-thiogalactopyranoside (IPTG) until the end of fermentation (˜24±1 h at 37° C.).

The fermentation monitoring platform is configured to record all conditions used to grow the His/NY-ESO-1-producing E. coli culture. The recorded parameters are: pH (regulated at 6.8 by addition of base), air/O2 usage, air flow and overpressure, temperature and feed rate. All cultures are performed in the presence of kanamycin (50 μg/mL).

m.3.2.S.2.2 Description of Manufacturing Process and Process Control

The final volume of cells is approximately 11 L depending on sample volumes collected throughout the process. At the end of fermentation, cells are harvested by centrifugation at 15,900×g for 30 min. At this stage, cell paste may be stored at −80° C. until further processing.

The feeding and induction phases at 37° C. are initiated immediately after the E. coli culture reaches an OD650 nm≧20 (typically after 14 h of fermentation at 28° C.).

In-process samples and harvest samples taken during the fermentation are examined by SDS-PAGE analysis, followed by Coomassie Blue dye staining. All culture media and buffers are sterilized by filtration and are free of materials from animal origin.

For fermentation lots produced according to this process, the obtained release testing data are presented in m3.2.S.4.4.

### Example 2

### Disruption of Cells and Extraction of Inclusion Bodies Containing HIS/NY-ESO-1

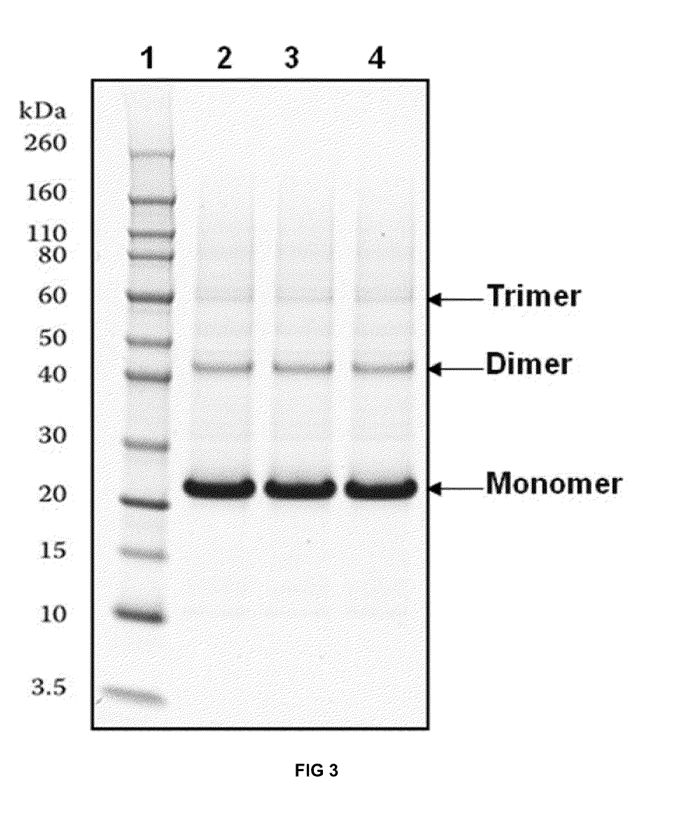

His/NY-ESO-1 protein is segregated into inclusion bodies (IB) during the induction phase. To release the IB, the E. coli BL21 B2270 cell paste obtained at the end of fermentation is further resuspended in chilled lysis buffer (50 mM Tris pH 8.0, 150 mM NaCl and 10 mM EDTA). The resuspended cells are disrupted by three passes through a high pressure homogenizer. After cell lysis, the inclusion bodies are harvested by centrifugation at 15,900×g for 30 min at 4° C. The resulting IB pellets are then washed with chilled wash containing 50 mM Tris pH 8.0 and 150 mM NaCl. A wash step can also be included as follows:
The insoluble fraction containing the inclusion bodies is recovered by centrifugation and the pellet washed one time with 20 mM Tris, 5% Triton X-100, pH 8.0
The insoluble fraction containing the inclusion bodies is recovered by centrifugation and the pellet washed twice with 20 mM Tris, 150 mM NaCl, pH8.0
   A flow chart summarizing this process is presented in FIG. 3.

### Example 3

### EXTRACTION and Purification of HIS/NY-ESO-1

The purification process is summarized in FIG. 3. It involves several purification steps:
Extraction of His/NY-ESO-1 protein from the washed IB pellets obtained after cell disruption (Section 2)
Immobilized metal ion (Ni²⁺) affinity chromatography (Ni²⁺-IMAC) procedure;
Conditioning and buffer exchange by gel filtration on a Sephadex G-25 column
Concentration and diafiltration by means of tangential flow diafiltration (TFF)
Sterile filtration
   Further details about each of these purification stages are provided in the following sections. m.3.2.S.2.2 Description of Manufacturing Process and Process Control See FIG. 3/4.

### m.3.2.S.2.2 Description of Manufacturing Process and Process Control

### 3.1. Description of Extraction and Purification Process

3.1.1. Extraction of His/NY-ESO-1 Protein from the Washed IB Pellets

Briefly, the washed IB pellets are resuspended in 20 mM Tris (pH 8.0), 5% N-lauroylsarcosine, 20 mM imidazole, 5 mM Tris (2-carboxyethyl) phosphine hydrochloride (TCEP). Solubilization is accomplished overnight by gentle agitation at room temperature. The extract is then clarified by centrifugation at 15,900×g for 30 min at 4° C. The supernatant is further filtered on a 0.45/0.22-μm filter capsule prior to affinity chromatography on a column containing immobilized Ni²⁺. The buffer composition used in this step is provided in m3.2.S.2.3.

### 3.1.2. Immobilized Ni²⁺ Affinity Chromatography (IMAC)

Antigen, His/NY-ESO-1, present in the resulting supernatant is captured and purified by direct injection on a nickel ion metal affinity resin (IMAC Sepharose 6FF). The resin is packed in a column pre-equilibrated in Tris 20 mM, pH 8.0 and then equilibrated in 20 mM Tris pH 8.0, 2% N-lauroylsarcosine, 20 mM imidazole, and 1 mM TCEP. After loading the solubilized IBs, the column is washed twice under the following conditions:
First wash is performed with a solution containing 20 mM Tris (pH 8.0), 1% N-lauroylsarcosine and 1 mM TCEP;
Second wash is performed with a solution containing 20 mM Tris (pH 7.0), 8 M urea, 0.5 M NaCl, 1 mM TCEP, and 20 mM imidazole.

His/NY-ESO-1 is eluted from the column using a buffer solution containing 20 mM Tris (pH 7.0), 8 M urea, 0.5 M NaCl, 1 mM TCEP, and 500 mM imidazole. The buffer composition used in this step is provided in m3.2.S.2.3.

### 3.1.3. Buffer Exchange

The buffer of His/NY-ESO-1-containing eluate is exchanged by passage through a Sephadex G-25 (medium) desalting column equilibrated with a 10 mM sodium acetate buffer (pH 5.0). The buffer composition used in this step is provided in m3.2.S.2.3.

### 3.1.4. Concentration and Diafiltration

The Sephadex G-25 flow-through containing the His/NY-ESO-1 fusion protein is then concentrated and diafiltered by means of a 10 kDa membrane against 10 mM sodium acetate buffer pH 5.0. The concentration of the final diafiltered material is adjusted to approximately 1.7 mg/mL in 10 mM sodium acetate buffer (pH 5.0). The buffer composition used in this step is provided in m3.2.S.2.3.

### m.3.2.S.2.2 Description of Manufacturing Process and Process Control

### 3.1.5. Sterile Filtration

Each individual batch of purified bulk is sterilized by filtration through a 0.45/0.22-μm cellulose acetate membrane. The purified bulk protein produced at this stage is designated as “Intermediate Purified Bulk” and is stored at −70° C. in 250 mL polyethylene terephthalate glycol (PETG).

### 3.2. In-Process Control

In-process testing of the purification process is performed on the samples indicated in Table 1 and includes:
Total protein content by the bicinchoninic acid (BCA) method;
UV measurement at λ=280 nm;
Electrophoretic profile by sodium dodecylsulphate polyacrylamide gel electrophoresis (SDS-PAGE) with Coomassie Blue staining analysis;
Identity of His/NY-ESO-1 by Western blotting analyses.
Bioburden

TABLE 1
In-process control of purification process

| | | BCA | UV | | Western | |
|---|---|---|---|---|---|---|
| Step | Sample^{1,2} | assay | 280 nm | SDSPAGE | blot | Bioburden |
| IB solubilization | Solubilized IB | X | | X | X | |
| Clarification | IMAC load | X | | X | X | |
| Ni2+-IMAC | Flow through | X | | X | X | |
| | Wash-out | X | | X | X | |
| | Eluate | X | | X | X | |
| Sephadex G-25 | Eluate | X | X | X | X | |
| Ultrafiltration- | Concentration | X | | X | X | |
| 10K | permeate | | | | | |
| | Diafiltration | X | — | | | |
| | permeate | | | | | |
| | Diafiltration | X | X | X | X | X |
| | retentate | | | | | |
| Filtration | Purified | X | | X | X | |
| | protein bulk | | | | | |

¹His/NY-ESO-1 is present in the following fractions: solubilized IB, Ni²⁺-IMAC eluate, Sephadex G-25 eluate, ultrafiltration retentate, and purified bulk.
²All samples taken are kept at −80° C. in sterile tubes.

### Characteristics of NY-ESO-1 Protein

192 amino acids recombinant protein with a pl of 9.1 (including His tag)
Theoretical MW of 19390.8 Da
5 Cys residues

### Characteristics of NY-ESO-1 Protein Solution Produced by Our Process

Soluble protein aggregates with hydrodynamic size of ≈22 nm
Concentration of ≈2 mg/mL
Weight averaged molecular weight of 640 kDa
Positive Zeta-potential of ≈23 mV
100% free cystein as determined by MS
Low complexity solution (10 mM Na-Ac pH 5.0)

### Multimeric Forms

SDS-PAGE profile ≈77% of monomer; 14% dimer; 2.5% trimer
   SDA-PAGE densitometry analysis was carried out using NuPAGE 4-12% Bis-Tris Gel;
   Coomassie Blue G-250; Reduced condition; and ImageQuant Software analysis. See FIG. 3/4 and the following table.

| | Lane (%) | | | | |
|---|---|---|---|---|---|
| NY-ESO-1 | P-036 | P-037 | P-038 | | CV |
| Bands | DNYEAHA005 | DNYEAHA006 | DNYEAHA007 | Mean | (%) |
| Trimer | 2.6 | 2.4 | 2.5 | 2.5 | 4.0 |
| Dimer | 13.9 | 14.6 | 13.6 | 14.0 | 3.7 |
| Monomer | 77.0 | 77.4 | 76.6 | 77.0 | 0.5 |
| Total [symbol] | 93.5 | 94.4 | 92.7 | 93.5 | 0.9 |

### Host Cell Protein Analysis.

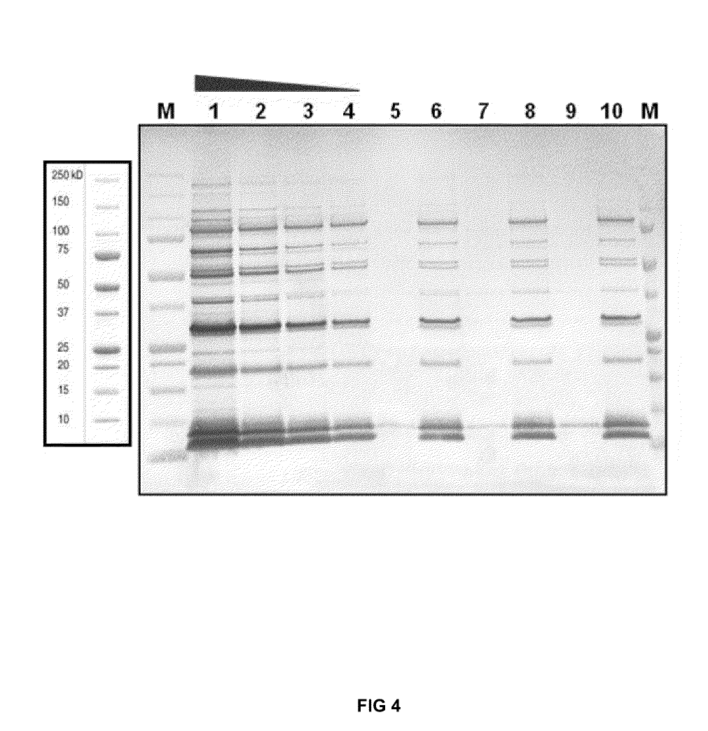

Host cell contaminants were investigated by Western blot using NuPAGE 4-12% Bis-Tris/Reduced. Polyclonal antibodies raised by standard techniques against E. coli BLR DE3 cellular extract were used to detect host cell protein. Host cell protein content in the NY-ESO-1 polypeptide produced in three separate runs using the disclosed process herein was determined to be no more than 5% w/w by densitometry analysis (lanes 5, 7, 9). See FIG. 4/4.

1. A process for purifying a NY-ESO-1 related polypeptide having an affinity tag comprising the steps of:
(a) solubilizing a composition comprising the polypeptide having an affinity tag in a buffer comprising N-lauroylsarcosine and Tris (2-carboxyethyl) phosphine hydrochloride (TCEP) to obtain a mixture; and
(b) purifying the mixture by immobilized metal affinity chromatography (IMAC) to obtain pure polypeptide having an affinity tag.

2. The process of claim 1 wherein the buffer comprises 20 mM Tris pH 8.0, 5% N-Lauroylsarcosine, 20 mM Imidazole, 5 mM TCEP.

3. The process of claim 1 wherein the composition is a cell pellet.

4. The process of claim 1 wherein the affinity tag is a metal ion binding affinity tag.

5. The process of claim 4 wherein the affinity tag is a his-tag.

6. The process of claim 1 wherein the step of purifying the mixture by IMAC further comprises the substeps of:
(i) loading the mixture on an IMAC substrate;
(ii) washing the substrate with a first buffer comprising N-Lauroylsarcosine and TCEP;
(iii) washing the substrate with a second buffer comprising Urea, NaCl, and TCEP;
(iv) eluting the polypeptide having an affinity tag with an elution buffer comprising Urea, NaCl, and TCEP to obtain pure polypeptide having an affinity tag.

7. The process of claim 1 wherein the IMAC is carried out using a Ni2+ substrate.

8. The process of claim 1 wherein the IMAC is carried out using substrate contained in a column.

9. The process of claim 6 wherein the first buffer comprises 20 mM Tris pH 8, 1% N-Lauroylsarcosine, 1 mM TCEP.

10. The process of claim 6 wherein the second buffer comprises 20 mM Tris pH 7, 8 M Urea, 0.5 M NaCl, 1 mM TCEP, and 20 mM imidazole.

11. The process of claim 6 wherein the elution buffer comprises 20 mM Tris pH 7, 0.8 M Urea, 0.5 M NaCl, 1 mM TCEP, 500 mM imidazole.

12. The process of claim 1 further comprising a step of subjecting the polypeptide having an affinity tag to buffer exchange following the step of purifying the mixture by IMAC.

13. The process of claim 12 wherein the buffer exchange step is carried out using chromatography on a sephadex G-25 column.

14. The process of claim 12 wherein the buffer exchange is carried out using a buffer comprising 10 mM sodium acetate, pH 5.

15. The process of claim 12 further comprising a step of concentrating and diafiltrating the polypeptide having an affinity tag following the step of buffer exchange.

16. The process of claim 15 wherein the concentrating and diafiltrating step is performed by TFF.

17. The process of claim 15 wherein the concentration and diafiltration is performed on a 10 kDa regenerated cellulose C-screen membrane in a buffer.

18. The process of any one of claim 15 wherein the concentration and diafiltration is done in a buffer comprising 10 mM sodium acetate, pH 5.

19. The process of claim 1 further comprising a step of clarifying the mixture following the step of solubilizing the composition.

20. The process of claim 19 wherein the clarification step further comprises centrifuging the mixture.

21. The process of claim 19 wherein the clarification step further comprises filtration of the mixture.

22. The process of claim 19 wherein the clarification step further comprises both centrifuging the mixture and filtration of the mixture.

23. The process of claim 20 wherein the centrifuging step occurs at 15,900×g.

24. The process of claim 21 wherein the filtration step occurs using a 0.45/0.22 um PES filter.

25. The process of claim 1 further comprising a step of disrupting cells comprising the polypeptide having an affinity tag prior to purifying the mixture by IMAC.

26. The process of claim 1 further comprising a step of subjecting the pure polypeptide having an affinity tag to filtration.

27. The process of claim 26 further wherein the filtration is sterile filtration.

28. The process of claim 26 wherein the filtration is done on a 0.45/0.2 Sartobran™ 300 cellulose acetate capsule.

29. A process for purifying a NY-ESO-1 related polypeptide having a his-tag comprising the steps of:
(a) disrupting host cells comprising the polypeptide having a his-tag by the substeps comprising
(i) suspending the host cells in a buffer comprising 50 mM Tris pH 8, 150 mM NaCl, 10 mM EDTA;
(ii) applying a cell disruption technique;
(iii) centrifuging the host cells at 15,900×g to obtain a pellet;
(iv) washing the pellet in a buffer comprising 50 mM Tris pH 8 and 150 mM NaCl;
(v) recovering the insoluble fraction containing the inclusion bodies by centrifugation and washing the pellet one time with 20 mM Tris, 5% Triton X-100, pH 8.0;
(vi) recovering the insoluble fraction containing the inclusion bodies by centrifugation and washing the pellet twice with 20 mM Tris, 150 mM NaCl, pH8.0;
(b) solubilizing the pellet in a buffer comprising 20 mM Tris pH 8.0, 5% N-Lauroylsarcosine, 20 mM Imidazole, 5 mM TCEP to obtain a mixture;
(c) clarifying the mixture by the substeps of:
(i) centrifuging the mixture at 15,900×g to obtain a supernatant;
(ii) filtration of the supernatant through a 0.45/0.22 um PES filter;
(d) purifying the mixture by IMAC comprising the substeps of:
(i) loading the mixture on a Ni2+-IMAC sepharose column;
(ii) washing the column with a buffer comprising 20 mM Tris pH 8, 1% N-Lauroylsarcosine, 1 mM TCEP;
(iii) washing the column with a buffer comprising 20 mM Tris pH 7, 8 M Urea, 0.5 M NaCl, 1 mM TCEP, 20 mM Imidazole;
(iv) eluting the polypeptide having a his-tag with a buffer comprising 20 mM Tris pH 7, 0.8 M Urea, 0.5 M NaCl, 1 mM TCEP, 500 mM imidazole to obtain an eluate comprising the polypeptide having a his-tag;
(e) subjecting the eluate to buffer exchange chromatography on a sephadex G-25 column using a buffer comprising 10 mM sodium acetate pH 5 to obtain an eluate comprising the polypeptide having a his-tag;
(f) concentrating and diafiltrating the polypeptide having a his-tag by TFF on a 10 kDa regenerated cellulose C-screen membrane in a buffer comprising 10 mM sodium acetate pH 5; and
(g) subjecting the polypeptide having a his-tag to sterile filtration on a 0.45/0.2 Sartobran™ 300 cellulose acetate capsule.

30. A composition comprising polypeptides, wherein said polypeptides comprise by w/w at least 95% monomer+dimer+trimer species of a polypeptide comprising the amino acid sequence of SEQ ID NO:1.

31. A composition comprising a polypeptide comprising the amino acid sequence of SEQ ID NO:1, further comprising no more than 5% host cell protein content.

32. A composition comprising a polypeptide comprising the amino acid sequence of SEQ ID NO:1, further comprising a low complexity solution.

33. The composition of claim 31 wherein the polypeptide comprises one or more of the following characteristics selected from the group consisting of:
(a) soluble polypeptide aggregates with hydrodynamic size of about 22 nm;
(b) concentration of at least about 2 mg/mL;
(c) weight averaged molecular weight of about 640 kDa;
(d) positive Zeta-potential of about 23 mV;
(e) 100% free of cystein as determined by MS; and
(f) low complexity solution comprising 10 mM Na-Ac pH 5.0.

34. The composition of claim 31 wherein the polypeptide forms about 77% of monomer; 14% dimer; and 2.5% trimer w/w as measured by SDS-PAGE.

35. A composition comprising N-Lauroyl sarcosine; TCEP; and one or more cancer testes antigens comprising full-length NY-ESO-1.

36. The composition of 35 wherein the full length NY-ESO-1 further comprises a fusion partner or a carrier protein.
